(19) United States
(12) Patent Application Publication
(10) Pub. No.: US 2010/0286765 A1
(43) Pub. Date: Nov. 11, 2010

(54) Medical Device Coatings and Coated Stents

(76) Inventor: Paul Zamecnik, Boston, MA (US)

Correspondence Address: Preti Flaherty, PO Box 9546, One City Center, Portland, ME, 04112-9546, US

(21) Appl. No.: 12/436,963

(22) Filed: May 7, 2009

(51) Int. Cl. A61F 2/06

(52) U.S. Cl. 623/1.46

(57) ABSTRACT

The invention provides a medical device coated with Ap₄A and/or an Ap₄A analog. In a preferred embodiment the medical device is a vascular stent.

### BACKGROUND OF THE INVENTION

1. Field of the invention

The invention relates to a medical device, e.g., a stent having drugs, agents or compounds affixed thereto to minimize a biological organism's reaction to the introduction of the medical device into the organism.

2. Summary of the Related Art

One of the many implantable medical devices used in the treatment of vascular disease is a stent. The stent is designed to prevent collapse of a vessel that has been weakened or damaged by angioplasty. Insertion of stents has been shown to prevent harmful remodeling of the vessel while healing of the vessel proceeds over several months after angioplasty.

Known stent designs include monofilament wire coil stents, welded metal cages and thin-walled metal cylinders with axial slots formed around the circumference. Known construction materials for use in stents include polymers, organic fabrics and biocompatible metals, such as stainless steel, silver, gold, tantalum, titanium, and shape memory alloys such as Nitinol.

Vascular stents are typically introduced percutaneously and transported intraluminally to a desired location within the vessel. The stent is then expanded within the vessel either mechanically, such as by the expansion of a balloon within the stent, or self-expanding by releasing stored energy upon actuation within the vessel.

Upon expansion of the stent during angioplasty, smooth muscle cells within the vessel wall become injured, initiating a thrombotic and inflammatory response. Contributing factors in this response include cell derived growth factors such as platelet derived growth factors, thrombin and other factors released by platelets. The resulting restenosis of the vessel is reduced through the permanent implantation of the stent, as apposed to balloon angioplasty alone. Nevertheless, blockage or even collapse of the stent may occur.

To reduce restenosis of the vessel in which the stent has been deployed, numerous drugs or agents have been utilized to coat the stent or to otherwise be diffusably released by the stent. For example, heparin coating of the stent has been shown to reduce restenosis. Other agents having anti-proliferative activity have been used, such as paclitataxel and rapamycin. These and other agents have been incorporated into bioabsorbable or biodegradable polymers as a coating on the stent, or have been applied to the surface of the stent through dip, spray, or spin coating processes, or by electrostatic abluminal coating. Unfortunately, many of the drugs or agents used for this purpose have their own associated toxicities and may therefore be less than optimally suitable.

More recently, doctors have found that a small number of patients develop blood clots within some drug coated stents long after they are implanted. Optimally a compound used to render a deployed stent less subject to restenosis should be sufficiently active to produce a therapeutic dose for an extended period of time, should not be toxic to the regrowth of endothelial cells within the vessel lumen, and should function to prevent blood clot formation within the stent.

The compound diadenosine 5′, 5′″, P¹, P⁴ tetraphosphate (Ap₄A) was first discovered by Moffatt, Can. J. Chem. 42: 599 (1964) as a minor by-product of the chemical synthesis of adenosine triphosphate (ATP). It was subsequently shown by Zamecnik et al., Biochem. Biophys. Res. Comm. 24: 91-97 (1966) to be present as the back reaction product in amino acid activation, the first step in protein synthesis, and to be a ubiquitous component of eukaryotic and prokaryotic cells. Flodgaard and Klenow, Biochem. J. 208: 737 (1982) discovered that platelets have a high content of Ap₄A in their dense granules.

Subsequent studies showed some promise for clinical applications for Ap₄A. Louie et al., Thromb. Res. 49: 557-565 (1988) showed that Ap₄A acts as a competitive inhibitor of ADP-induced platelet aggregation and inhibited clot formation in a rabbit model. However, Ap₄A was found to have a short in vivo half life due to phosphodiesterase activity. Consequently, analogs of Ap₄A were developed and studied. Zamecnik et al., Proc. Natl. Acad. Sci. USA 89: 2370-2373 (1992) teaches that Ap₄A analogs having a halogenated methylene bridge in place of an oxygen atom are superior to Ap₄A in inhibiting ADP-induced aggregation of human platelets and are resistant to hydrolytic enzymes. Chan et al., Proc. Natl. Acad. Sci. USA 94: 4034-4039 (1997) discloses additional Ap₄A analogs that are effective at preventing platelet aggregation induced by ADP and other agonists.

### BRIEF SUMMARY OF THE INVENTION

The invention provides medical devices coated with Ap₄A and/or Ap₄A analogs, either alone or in combination with other medicaments. The medical devices according to the invention are expected to prevent restenosis and thrombus formation associated with some drug coated stents.

### DETAILED DESCRIPTION OF THE PREFERRED EMBODIMENTS

The invention provides medical devices coated with Ap₄A and/or Ap₄A analogs, either alone or in combination with other medicaments. The medical devices according to the invention are expected to prevent restenosis and thrombus formation associated with some drug coated stents.

The patents and publications cited herein reflect the level of knowledge in the field and are hereby incorporated by reference in their entirety. Any conflict between the teachings of these references and this specification shall be resolved in favor of the latter.

In a first aspect, the invention provides a vascular stent that is coated with Ap₄A and/or Ap₄A analogs.

For purposes of the invention, the term “stent” is given its conventional meaning within the medical devices art and includes both mechanically expandable and self-expandable stents.

For purposes of the invention, the term “coated with” means that the stent is associated with Ap₄A or a Ap₄A analog in a manner that allows the Ap₄A or an Ap₄A analog to locally contact platelets and other cell types that are in contact with, or in the immediate vicinity of the stent. Such associations include covalent attachment of the molecule to the stent. Such covalent attachment may be reversible or irreversible. In preferred embodiments, reversible attachment to the stent may be via a chemical linkage that is labile in vivo. Such labile linkages include ester linkages, including phosphoester linkages, as well as amide or peptide linkages.

Many means of coating the stent are known in the art, including dip, spray or spin coating processes. For example, US2006/0217801A1 discloses a stent having an engineered abluminal surface that is engineered to have a material comprising a mixture of a polymer and a drug extending from the abluminal surface by depositing microdrops of the mixture onto the abluminal surface of the stent. US2006/0229706A1 discloses a stent having a drug-release coating of a thickness of 3-30 microns and composed of 20-80 weight percent polymer substrate and 20-80 weight percent active compound, where the coating is effective to release an amount of the active compound to prevent restenosis at the site of the stent. In a preferred embodiment, the stent body is metallic and the polymer is selected from polymethylacrylate, ethylene vinyl alcohol, poly-lactide polymers (especially poly-dl-lactide), ε-caprolactone, ethyl vinyl acetate, polyvinyl alcohol and polyethylene oxide. US2006/0222756A1 discloses a method for coating a stent with a therapeutic agent comprising the steps of creating a polymer utilizing vinylidene fluoride and hexafluoropropylene in a batch emulsion polymerization process, priming the stent with the polymer utilizing a dip coating process, creating a polymer and therapeutic agent mixture, applying the polymer and therapeutic agent mixture on the primer layer utilizing a spin coating process, and drying the coated stent in a vacuum oven for approximately sixteen hours at a temperature in the range of fifty to sixty degrees centigrade. US2006/0216431A1 discloses a method for electrostatically coating the abluminal surfaces of a stent that is crimp-mounted on a balloon catheter comprising threading a wire through a lumen of the stent-balloon assembly and applying a charge to the wire, while the stent is grounded, and applying an electrostatic spray coating to the stent-balloon assembly. Alternatively, a charge can be applied to the stent that is opposite to the charge applied to the wire. In a preferred embodiment, the wire is the guidewire for the catheter which is threaded through the guidewire lumen. Generally, the delivery of an active agent from the coating to the vascular wall is through diffusion of the active agent through either a bulk polymer or through pores that are created in the polymer structure, or by erosion of a biodegradable coating.

For purposes of the invention, Ap₄A refers to diadenosine 5′, 5′″, P¹, P⁴ tetraphosphate, which has the structural formula (I):

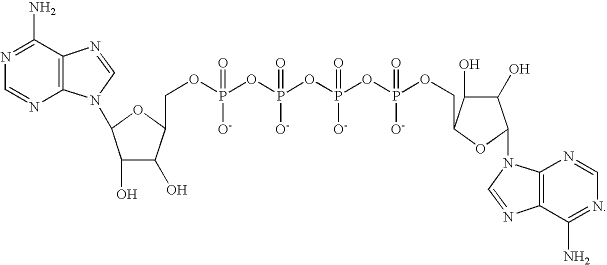

For purposes of the invention, an “Ap₄A analog” is a compound having anti-platelet activity similar to or greater than Ap₄A, and a structural formula derived from (I), wherein one or more atom is replaced by another atom or atoms or one or more chemical bond. For example, one or more bridging oxygen atoms in formula (I) may be replaced by a methylene, halomethylene, or dihalomethylene group. One or more non-bridging oxygen atom may be replaced by a sulfur atom, by a lower alkyl group (including a methyl group), by an O-lower alkyl group (including an O-methyl group), by an amino or amido group, or by a boronate group. One or more phosphate group may be replaced by a peptide linkage. One or both hydroxyl moieties on either or both ribosyl groups may be replaced by hydrogen, or by halo or alkoxy groups, or may be in a locked 2′,4′ ring configuration. One adenine moiety may be missing or replaced by purine or another purine analog, or by pyrimidine or another pyrimidine analog. One adenosyl moiety may be missing. These modifications, or any combination of these modifications are within the intended scope of an “Ap₄A analog”, as long as the resultant compound exhibits an anti-platelet activity similar to or greater than Ap₄A. “Similar to” means at least about one tenth the anti-platelet activity of Ap₄A in a conventional anti-platelet assay, e.g., as taught in Chan et al., Proc. Natl. Acad. Sci. USA 94: 4034-4039 (1997).

Ap₄A or an Ap₄A analog should be present on the stent or in the stent coating in an amount effective to prevent platelet aggregation on a surface of the stent or within the stent.

Those skilled in the art will recognize that the present invention can be used alone or in conjunction with other medicaments to coat the stent.

What is claimed is:

1. A medical device coated with Ap₄A and/or an Ap₄A analog.

2. The medical device according to claim 2 which is a vascular stent.
